(19) United States
(12) Plant Patent Application Publication
(10) Pub. No.: US 2003/0014790 P1
(43) Pub. Date: Jan. 16, 2003

(54) Peach tree named 'GL4/66'

(76) Inventors: Paul G. Glucina, Mt. Albert (NZ); Michael T. Malone, Havelock North (NZ)

Correspondence Address: Michelle Bos, Stratton Ballew PLLC, 213 South 12th Avenue, Yakima, WA, 98902, US

(21) Appl. No.: 09/905,574

(22) Filed: Jul. 12, 2001

(51) Int. Cl. A01H 5/00

(52) U.S. Cl. PLT/195

(57) ABSTRACT

A new and distinct variety of peach tree (Prunus persica) named ‘GL4/66,’ a selection derived from the open pollinated seed of ‘Yumyeong’ (not patented), is described. The new variety is distinguishable from the parent variety by its early harvest maturity, two weeks earlier than ‘Yumyeong,’ and by its sweeter, more aromatic fruit. A further distinction between ‘GL4/66’ and ‘Yumyeong’ is the higher chill requirement of the new variety. Its novelty consists of a unique combination of features that include late flowering, heavy and regular cropping, larger very firm, white crisp-textured, clingstone fruit with a pale pink overcolour. The fruit have a sweet, sub-acid flavor with very good fruit aroma.

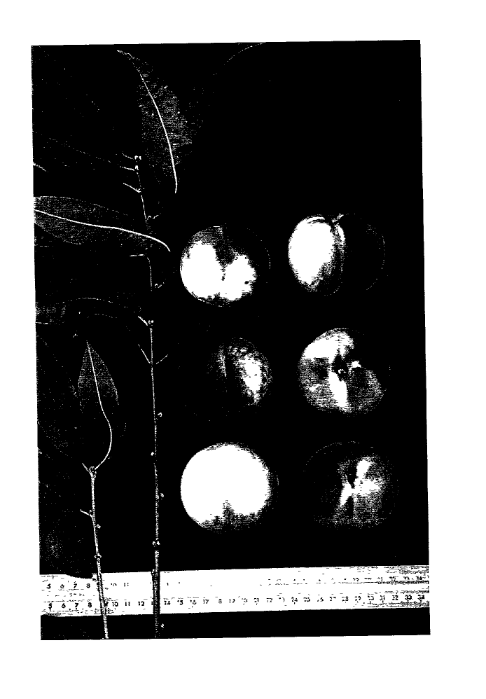

### ORIGIN OF THE VARIETY

The present new and distinct variety of peach tree (Prunus persica) was originated from an open pollinated population of seedlings derived from the variety ‘Yumyeong’ planted in 1990 at the HortResearch experimental orchard in Havelock North, New Zealand. Paul G. Glucina established a large group of open-pollinated seedlings on their own roots and under careful observation one seedling representing the present variety was selected and asexually propagated for further evaluation by Michael T. Malone. The selection was further propagated by asexual reproduction for the first time in 1995.

### ASEXUAL REPRODUCTION OF THE VARIETY

The new variety was budded onto ‘Golden Queen’ peach rootstock and shows the unique combination of characteristics and distinctive colour are true to the original seedling tree and are transmitted through succeeding asexual generations.

### SUMMARY OF THE VARIETY

The described variety of peach tree is of a medium to large size, vigorous, with a spreading growth habit. It is a regular and productive bearer of medium to large white flesh, clingstone fruit with a sweet sub-acid taste and very firm crisp flesh with good fruit aroma. The fruit has very good flavour and eating quality. The firm flesh exhibits good storage and the skin has an attractive pink colour over a cream background. The fruit ripens mid season approximately two weeks before the ‘Yumyeong’ parent variety.

### BRIEF DESCRIPTION OF THE DRAWINGS

The accompanying photograph shows typical specimens of the foliage and the fruit of the new variety as depicted in colours as nearly true as is reasonably possible in a colour representation of this type. The upper and lower surfaces of the leaves, and the exterior view of the fruit, are shown. The photograph was taken after two weeks air storage of fruit harvested at eating maturity.

### DETAILED DESCRIPTION OF THE NEW VARIETY

The following is a detailed description of the new variety with colour terminology in accordance with The Royal Horticultural Society Colour Chart, 1986 edition. The specimens described were grown in Havelock North and Clyde, Central Otago, New Zealand. The observations were made in the 2001 season on trees which were four years old at the time.

Tree:

Size.—Large.

Vigour.—Vigorous.

Form.—Pruned to central leader, with a spreading growth habit.

Trunk:

Size.—Medium.

Texture.—Medium rough.

Colour.—Grey/brown 197A.

Branch:

Size.—Medium.

Lenticels.—Medium number, large elongated shape.

Leaves:

Size.—Medium to large.

Average width.—55 mm.

Average length.—190 mm.

Form.—Pointed.

Shape of tip.—Acuminate.

Angle of tip.—Narrow acute.

Shape of base.—Attenuate.

Incision of margin.—Crenate.

Thickness.—Medium.

Surface.—Smooth.

Petiole.—Medium length, medium thickness (average 15 mm).

Glands.—Reniform, elongated.

Nectaries on fully developed leaf.—Usually more than two, average 4.

Colour.—Upper Surface Dark green 141A.

Color.—Lower Surface Light green 146B.

Flower Buds:

Size.—Medium to large.

Length.—Medium.

Form.—Plump.

Flowers:

Size.—Medium to large.

Pollen.—Present.

Colour.—Medium pink.

Petal shape.—Round.

Pubescence of ovary.—Present.

Fruit:

Maturity for consumption.—Ripe.

Season.—Mid-late.

Size.—Medium.

Average weight.—170 g.

Average diameter across suture line.—73 mm.

Average length.—64 mm.

Shape (ventral view).—Round oblate, symmetrical.

Shape of pistil end.—Rounded, slightly depressed.

Suture.—Shallow, extends from base to apex.

Cavity.—Rounded, medium to deep, average depth 9 mm, average width 28 mm.

Flesh:

Ripening.—Even.

Texture.—Firm, non-melting.

Fibres.—Non-fibrous.

Aroma.—High.

Eating quality.—Good.

Flavour.—Low acid, mild.

Soluble solids.—Average 10-12° Brix.

Juice.—Moderately juicy.

Colour.—White 155D.

Anthocyanin.—Low.

Stem:

Size.—Medium, average 0.5 cm.

Colour.—Yellow green 150C.

Skin:

Thickness.—Medium.

Texture.—Medium to strong adherence of skin to flesh.

Density of pubescence.—Medium.

Tendency to crack.—None.

Colour.—Red 46A, blending to dark pink 48A blush pattern of over colour. Cream ground colour 11D.

Stone:

Type.—Clingstone.

Size.—Medium.

Average length.—32 mm.

Average width.—26 mm.

Average thickness.—18 mm.

Average weight.—6.5 g.

Shape (lateral view).—Round/elliptic.

Surface.—Pits and grooves, pitted towards the base.

Tendency to split.—None.

Colour.—Brown 165A.

Use.—Local and export markets.

Storage.—Good; storage trials indicate 2-3 weeks storage without internal breakdown of flesh.

The present new variety of peach tree, its flowers, foliage and fruit herein described may vary slightly in detail due to climate, soil conditions and cultural practices under which the variety may be grown. The present description is that of the variety grown under the environmental conditions prevailing near Havelock North, Hawke's Bay, New Zealand.

We claim:

1. A new and distinct variety of peach tree named ‘GL4/66,’ including all its parts and the fruit thereof, substantially as illustrated and described.
